(19) United States
(12) Patent Application Publication
(10) Pub. No.: US 2020/0159748 A1
(43) Pub. Date: May 21, 2020

(54) System or a Method of Medical Code Recommendation Using Background Knowledge of Code Assignment Patterns

(71) Applicant: ezDI Inc., Louisville, KY (US)

(72) Inventors: Nehal Shah, Louisville, KY (US); Suhas Indirakshan Nair, Ahmedabad (IN); Raxitkumar Vishnupuri Goswami, Vadali (IN); Krunal Prahladbhai Patel, Gandhidham (IN)

(21) Appl. No.: 16/659,522

(22) Filed: Oct. 21, 2019

Related U.S. Application Data
(60) Provisional application No. 62/749,097, filed on Oct. 22, 2018.

(51) Int. Cl. G06F 16/28; G16H 70/00; G06F 16/248; G06N 5/02

(52) U.S. Cl. CPC G06F 16/285; G06N 5/02; G06F 16/248; G16H 70/00

(57) ABSTRACT

A method of code recommendation includes analysing/tracking a pattern of search term and corresponding code assignment by the user, suggesting/recommending previously assigned codes when the same query is made subsequently, suggesting the co-existence of codes, and analysing/tracking the pattern of code co-existence assigning multiple codes.

### TECHNICAL FIELD

The present invention relates to a system and a method for code recommendation, and more particularly to the use of encoder in a CAC system or standalone encoder for recommending appropriate code(s) to the user as per the search query.

### BACKGROUND

Currently there are two types of encoders available:

1) Book/knowledge-based encoder—A knowledge-based encoder provides the official code sets as they are published by the cooperating parties, which assist the user in selecting the appropriate code. This can be a more efficient option for experienced users who know what they are looking for and the appropriate terms to use. This encoder utilizes a coding process that is determined by each coder's preferences and experience. This technique offers greater ease, accuracy and familiarity to coders who are trained using the code book, promotes the proper use of coding conventions, which supports good coding practices, and provides instructional notes and access to resources at the point of coding; and

2) Logic based encoder that guides the user to a code through a series of simple questions, each based on the preceding answer. This type of encoder uses a coding process that is determined by the software's proprietary logic and design; and assigns codes based on the user's responses to a series of questions, thus allowing the coders to collect multiple associated codes in one session.

These encoders are unable to track the code assignment patterns i.e every time the coder enters a search query, he/she has to follow the same procedure over and over again. It becomes very tiresome and time-consuming for the coder to repeat the same procedure every day.

Moreover, the encoders used currently cannot identify the group of codes coded together. Therefore, using the encoder integrated with artificial intelligence, tracks the coder behaviour and based on statistical analysis recommends the codes that are frequently coded together.

Currently, the coders either use logic or book based encoders and there is no solution available to

1) to integrate the best of book based and logic based encoder

2) provide the assignment of multiple codes together with a single search.

Another advantage of the present invention is that the pattern between the search term query and code assignment is tracked across all the hospitals and coders using the ezCAC (ez computer assisted coding) system. As a result there is a lot of data available to make a correlation between the search terms used and the corresponding codes assigned. Obviously, the data can be used or extrapolated for many other purposes as well.

1) In addition to the above, this invention solves the problem of searching the same term multiple times. Since this invention has stored the data as a correspondence between the searched term and the assigned code, the user need not go through the process of searching the same term multiple times.

2) The present invention also identifies the group of codes that are coded together.

### SUMMARY

The present invention provides a system and a method of code recommendation; more particularly, it relates to the method of code recommendation using artificial intelligence integrated with computer assisted coding. The system consists of different components as labelled in FIG. 1). For easy understanding of the procedure and the layout, FIG. 1) is divided into two parts—the front end or the user interface and the back end. The front end (A) of the system displays the interactive applications codebook and Computer assisted coding (CAC). The back end (B) comprises of the various components like the case databases and various processing components. Each of the components and its function is described below in detail.

The component (1) is the user interface consisting of the codebook and the CAC applications. It is on this interface that the user inputs the query in the form of term abbreviation or code. Once the code is assigned by the coder using CAC, it is lodged to the data pipeline for billed codes (3). This is an async function that keeps the billed codes in the queue ready to be sent to the Recommendation knowledge processing system (4). This component is triggered when a case is completed and billed. Once this is done the whole case data is uploaded to the data cloud in the code recommendation data (5). This forms a part of the central database of the code recommendation system. This is in turn linked to the user interface. As a result when the user searches the term or code, this database searches the codes that are co-assigned with the said code. These recommended codes are ranked as per the co-occurrence percentage. The output also shows the ranks of the codes according to the hospital.

FIG. 2 is the screenshot of the search term and the corresponding codes and the assigned codes obtained. In this example, we have searched the term ‘htn’ and the tab below shows the codes with the letters htn. On the right are codes that are frequently coded together with htn along with the hospital and the percentage of the corresponding code. The recommended codes are ranked on the basis of percentage of co-occurrence.

This invention also enables the user to assign multiple codes in a single time.

This invention allows the tracking of search term query and corresponding assignment on a fairly large scale. Moreover, it utilizes a lot of data that is not being used. This will open many more opportunities to use this data in different ways. Those skilled in the art will realize the application of the using and extrapolating this code assignment data in many different ways.

### BRIEF DESCRIPTION OF DRAWINGS

FIG. 1 is the procedural flowchart of the code recommendation system

FIG. 2 is an embodiment of the user interface display

### DETAILED DESCRIPTION OF DRAWINGS

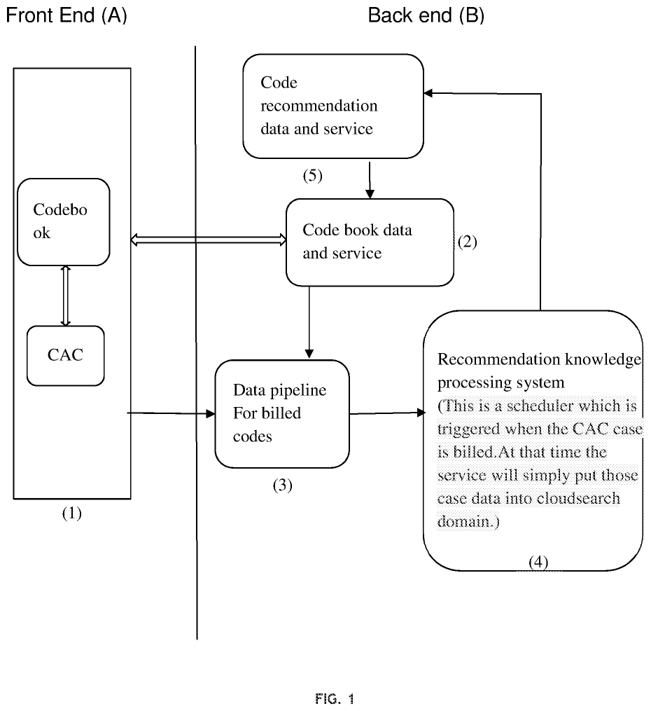

FIG. 1 is the procedure flowchart for the code recommendation system. The numbers mentioned in parentheses represent the individual parts of the code recommendation procedure. The procedure drawing is marked as front end/user interface and backend for the easy understanding.

The tab (1) represents the user interface that features the codebook and CAC application. This tab is connected to the codebook data and codebook search service (2) where the code data is stored. This database (2) is in turn connected to the data pipeline for billed codes. The function of this data pipeline is to queue the codes and transfer to the recommendation knowledge processing service (4) that is triggered when a CAC case is completed and billed. Once the case is complete the system will upload the case data on the cloud search domain, i.e. the code recommendation data (5) for further action. Once the case data is uploaded to the code recommendation data (5), it further connects to the code book data and service from where the data can be displayed on the user interface.

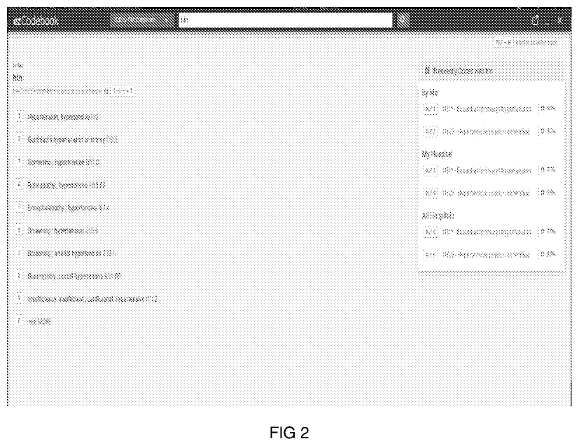

FIG. 2 is the screenshot of the search term and the corresponding codes and the assigned codes obtained. In this example, we have searched the term ‘htn’ and the tab below shows the codes with the letters htn. On the right are codes that are frequently coded together with htn along with the hospital and the percentage of the corresponding code. The recommended codes are ranked on the basis of percentage of co-occurrence.

### DETAILED DESCRIPTION OF INVENTION

One embodiment of the present invention provides a method of code recommendation which includes tracking the pattern of search term and corresponding code assignment by the user; and recommending the previously assigned codes when the same query is made subsequently. This is done by the use of artificial intelligence integrated computer assisted coding software. The code assignment data is collected from all the ezCAC users and the statistical analysis obtained is used to rank the recommended codes. The code assignment data is present in the data cloud and is harnessed from the cloud.

The data on the ezCAC platform is used to suggest co-existence of codes, i.e. if two or more codes are frequently coded together, the code recommendation system recommends the other codes when one of the codes is assigned. The detailed steps are as follows:

Once the coder assigns the code from the tabular type ezdi codebook, 5 other codes are recommended that are concurrently coded with the said assigned code. The recommendation of these five codes is based on the statistical analysis of the data at the backend. The data management is done at the backend. Moreover, the codes are ranked based on highest co-occurrence with the code assigned. The co-occurring codes are found by matching parameters between the current case and historical cases, based on the parameters like-patient class, patient gender, Patient age group (for example 0-3, 4-17, 18-50, 51-100), etc. The list is non exhaustive. Additionally, the coder can select multiple codes for assignment. This feature is currently not available with any of the coding systems. Mostly, the systems available currently are focused on integrating the various dictionary sources, policy documents, etc.

It is contemplated that the code recommendation system that is a part of ezCAC can be used on the desktop computer, laptop or any standard operating system.

1. A method of code recommendation for suggesting associated codes comprising the steps of:
(a) tracking search queries received from one or more users and corresponding assignments of a code selected from a list of search results presented to the respective users in response to each search query;
(b) suggesting a code previously assigned following receipt of a particular search query when the particular search query is received subsequently based on the tracked search queries and corresponding code assignments;
(c) tracking assignments of groups of associated codes by the one or more users;
(d) suggesting a group of a plurality of potential associated codes for an assigned code based on the tracked assignments of groups of associated codes.

2. A method of claim 1, wherein the pattern is tracked across all the users using this system.

3. A method of claim 1, wherein the code recommendation system is auto updated every time a case is coded using this system.

4. A method of claim 1, wherein the recommended codes are ranked based on the frequency of coexistence of codes.

5. A method of claim 1, wherein the codes are recommended based on user's pattern of searching and assigning codes.
